(19) United States
(12) Patent Application Publication
(10) Pub. No.: US 2021/0398689 A1
(43) Pub. Date: Dec. 23, 2021

(54) AUTONOMOUS MAPPING AND MONITORING POTENTIAL INFECTION EVENTS

(71) Applicant: CORSIGHT.AI, Tel Aviv (IL)

(72) Inventors: Karina Odinaev, Tel Aviv (IL); Matan NOGA, Tel Aviv (IL)

(73) Assignee: CORSIGHT.AI, Tel Aviv (IL)

(21) Appl. No.: 17/304,651

(22) Filed: Jun. 23, 2021

Related U.S. Application Data
(60) Provisional application No. 62/705,355, filed on Jun. 23, 2020.

(51) Int. Cl. G16H 50/80; G06K 9/00; G06K 9/62

(52) U.S. Cl. CPC G16H 50/80; G06K 9/00288; G06K 9/00778; G06K 9/6215; G06K 9/00362; G06K 9/00335; G06K 9/00677

(57) ABSTRACT

Systems, and method and computer readable media that store instructions for monitoring potential infection events.

### BACKGROUND

In the recent years we have experienced many outbursts of highly infectious diseases. A single sick person may infect tens of people that may, in turn, infect many other people.

Various smart-phone based applications use the inaccurate GPS-based locations of the smart-phones to monitor the locations of their owners.

Nevertheless—the accuracy of the GPS is not adequate (especially in urban area) to provide an indication about the proximity between persons especially in high traffic/dense venue.

There is a growing need to monitor persons involved in potential infection events.

### SUMMARY

There may be provided systems, methods, and computer readable medium as illustrated in the specification.

### BRIEF DESCRIPTION OF THE DRAWINGS

The embodiments of the disclosure will be understood and appreciated more fully from the following detailed description, taken in conjunction with the drawings in which:

FIG. 1 illustrates an example of a method;

FIG. 2 illustrates an example of a system;

FIG. 3 illustrates a first suspected person, other suspected persons, and a non-suspected person;

FIG. 4 illustrates sensors, a first suspected person, other suspected persons, and a non-suspected person; and

FIG. 5 illustrates an asset, an access control entity, a first suspected person, other suspected persons, and a non-suspected person.

### DESCRIPTION OF EXAMPLE EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a device or system capable of executing the method and/or to a non-transitory computer readable medium that stores instructions for executing the method.

Any reference in the specification to a system or device should be applied mutatis mutandis to a method that may be executed by the system, and/or may be applied mutatis mutandis to non-transitory computer readable medium that stores instructions executable by the system.

Any reference in the specification to a non-transitory computer readable medium should be applied mutatis mutandis to a device or system capable of executing instructions stored in the non-transitory computer readable medium and/or may be applied mutatis mutandis to a method for executing the instructions.

Any combination of any module or unit listed in any of the figures, any part of the specification and/or any claims may be provided.

The specification and/or drawings may refer to an image. An image is an example of a media unit. Any reference to an image may be applied mutatis mutandis to a media unit. A media unit may be an example of sensed information unit. Any reference to a media unit may be applied mutatis mutandis to sensed information. The sensed information may be sensed by any type of sensors—such as a visual light camera, or a sensor that may sense infrared, radar imagery, ultrasound, electro-optics, radiography, LIDAR (light detection and ranging), etc.

The specification and/or drawings may refer to a processor. The processor may be a processing circuitry. The processing circuitry may be implemented as a central processing unit (CPU), and/or one or more other integrated circuits such as application-specific integrated circuits (ASICs), field programmable gate arrays (FPGAs), full-custom integrated circuits, etc., or a combination of such integrated circuits.

Any combination of any steps of any method illustrated in the specification and/or drawings may be provided.

Any combination of any subject matter of any of claims may be provided.

Any combinations of systems, units, components, processors, sensors, illustrated in the specification and/or drawings may be provided.

The analysis of content of a media unit may be executed by generating a signature of the media unit and by comparing the signature to reference signatures. The reference signatures may be arranged in one or more concept structures or may be arranged in any other manner. The signatures may be used for object detection or for any other use.

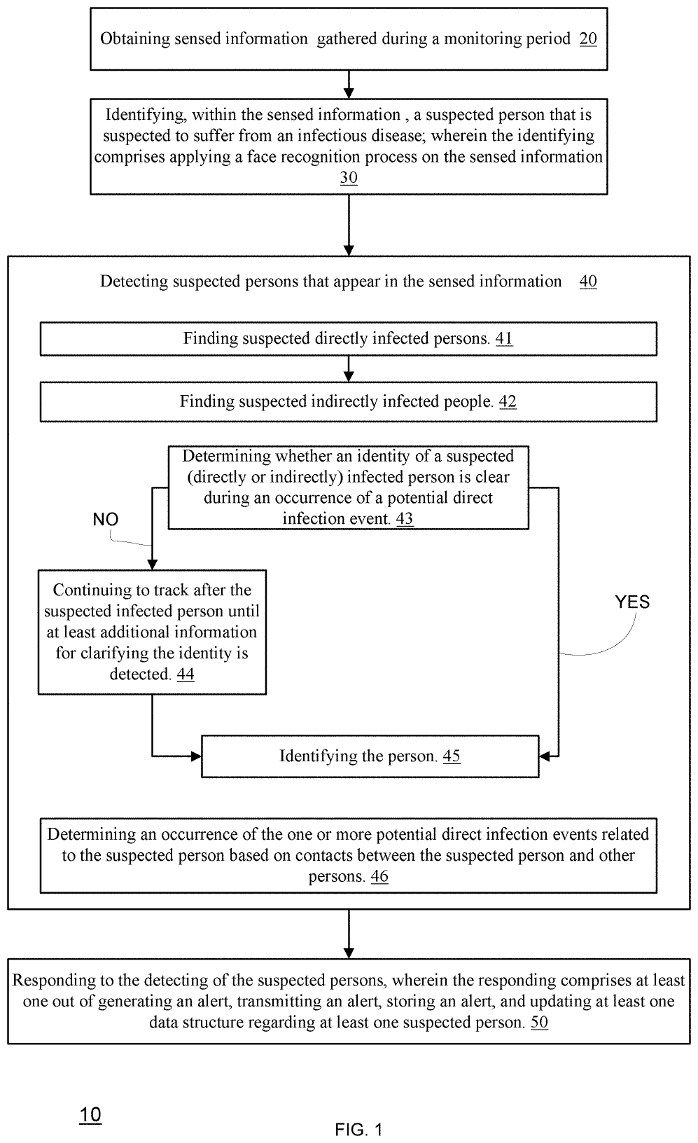

FIG. 1 illustrates method 10 for monitoring potential infection events.

Method 10 may start by step 20 of obtaining sensed information gathered during a monitoring period.

The sensed information may be visual information, thermal sensed information, infrared sensed information, and the like.

Step 20 may be followed by step 30 of identifying, within the sensed information, a first suspected person that is suspected to suffer from an infectious disease; wherein the identifying comprises applying a face recognition process on the sensed information.

Step 30 may be followed by step 40 of detecting suspected persons that appear in the sensed information.

Step 40 may include step 41 of finding suspected directly infected persons, and step 42 of finding suspected indirectly infected people.

Step 40 may be followed by step 50 of responding to the detecting of the suspected persons, wherein the responding comprises at least one out of generating an alert, transmitting an alert, storing an alert, and updating at least one data structure regarding at least one suspected person.

A suspected directly infected person was involved in a suspected direct infection event related to the suspected person. Step 41 may include finding of the suspected directly infected persons is based on distances between the first suspected person and other persons.

An indirectly infected people was involved in one or more other suspected infection events that are associated with the suspected direct infection events.

The association may be direct or indirect. A suspected indirect infected person may be involved in a suspected infection event with another person. The other person may be a suspected direct infected person or another suspected indirect infected person.

The finding of each one of steps 41 and 42 may be based on inter-person distances and timing periods in which the inter-person distances were maintained.

The method may also include determining a severity of a suspected infection event. The determining of the severity may be executed in any manner—for example by considering other parameters such as—was the infected person/person in proximity was wearing a mask or not.

The method may also include determining a probability of infection resulting from a suspected infection event. The probability may be calculated in various manners—for example based on distance between persons, and/or duration of maintaining the distance between persons.

When first detecting any suspected person—his identity may be clear or not. For example—if the face of the suspected person is images at the time of first detection—then face recognition may assist in detecting the person. If not (for example only a rear view of the person if acquired at the time of first detection)—the method may continue tracking after the person until his identity is clear—for example waiting until a detailed enough image of the face of the person is acquired.

Step 40 may include step 43 of determining whether an identity of a suspected (directly or indirectly) infected person is clear during an occurrence of a potential direct infection event.

If yes—jumping to step 45.

If no—step 43 may be followed by step 44 of continuing to track after the suspected infected person until at least additional information for clarifying the identity is detected.

Step 44 is also followed by step 45.

Step 45 include identifying the person.

Steps 43, 44 and 45 may be executed for each of the suspected infected persons.

Step 41 may also include step 46 of determining an occurrence of the one or more potential direct infection events related to the first suspected person based on contacts between the first suspected person and other persons.

Step 20 may include obtaining the sensed information from multiple sensors. For example—different cameras distributed in different locations.

The method according to claim 1 wherein the monitoring and the detecting is based on generating signatures of the sensed information.

Steps 20, 30, 40 and 50 may be executed in real time (for example within less than a second, within up to a minute, up to a few minutes, and the like).

Step 50 may include generating and transmitting, in real time, alerts that notify people located at a vicinity of the suspected persons, about the suspected persons.

When the method is executed in real time—there may be provided a database of sick people. When the method identified a sick person, based on the database, the method may generate an alert in real time—to notify people in the vicinity of the sick person.

Step 50 may include generating and transmitting, in real time, alerts that notify at least one access control entity (human guard, automatic gate control, and the like) of at least one asset that is located at a vicinity of at least one suspected person, about the at least one suspected person. This will allow to restrict access of persons to the asset.

The method according to claim 1 wherein the obtaining, identifying, detecting, and responding are executed in non-real time and wherein the responding comprises analyzing a distribution of a pandemic.

Step 30 and/or 40 or any object related identification and/or any movement analysis can be done in various manners—by applying a deep neural network, by applying a machine learning process, by generating signatures of the images, by face recognition algorithms, and the like. An example of a signature generation process and/or object detection is illustrated in U.S. patent application Ser. No. 16/542,327 which is incorporated herein by reference.

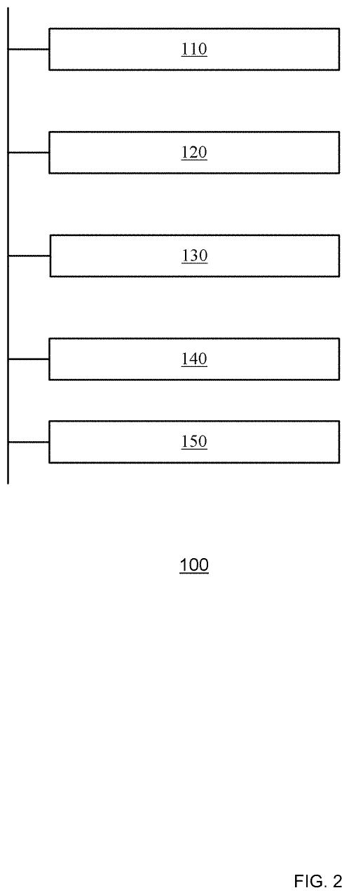

FIG. 2 illustrates a computerized system 100. Computerized system 100 may include sensed information gathering unit 110, processor 120, response unit 130, memory unit 140 and input/output unit 150.

The processor 120 and/or the input/output unit 140 and/or memory unit 150 may perform at least some parts of the functions of the sensed information gathering unit 110.

The processor 120 and/or the input/output unit 140 and/or memory unit 150 may perform at least some parts of the response unit 130.

The sensed information gathering unit 110 that is configured to gather sensed information during a monitoring period. The sensed information gathering unit 110 may store the sensed information and/or may send the sensed information to the memory unit 140.

The sensed information gathering unit 110 may include one or more sensors. Additionally or alternatively, the sensed information gathering unit 110 may retrieve sensed information from the sensors or from any other source of sensed information (including databases).

The processor 120 may be configured to identify, within the sensed information, a first suspected person that is suspected to suffer from an infectious disease. The identifying comprises applying a face recognition process on the sensed information.

The identity of the first suspected person may be stored in a database of suspected persons. For example—a fever database that stores information about persons that have fever. The database may be a governmental database, a hospital database, or any other database.

Additionally or alternatively, the processor may perform sensed information analysis that may determine whether a person is suspected—for example if the sensed information is indicative of fever or any externally noticeable symptom. The identification may include comparing the skin tint variability of the same person at different points of time.

The processor 120 may be configured to detect suspected persons that appear in the sensed information. The detecting may include finding suspected directly infected persons and finding suspected indirectly infected people.

The response unit 130 may be configured to respond to the detecting of the suspected persons. The responding may include at least one out of generating an alert, transmitting an alert, storing an alert, and updating at least one data structure regarding at least one suspected person.

Input output unit 140 may receive and/or output information, and/or alerts and/or reports, and the like. Input output unit 140 may be any suitable communications component such as a network interface card, universal serial bus (USB) port, disk reader, modem or transceiver that may be operative to use protocols such as are known in the art to communicate either directly, or indirectly, with other elements of system 100 and/or other entities.

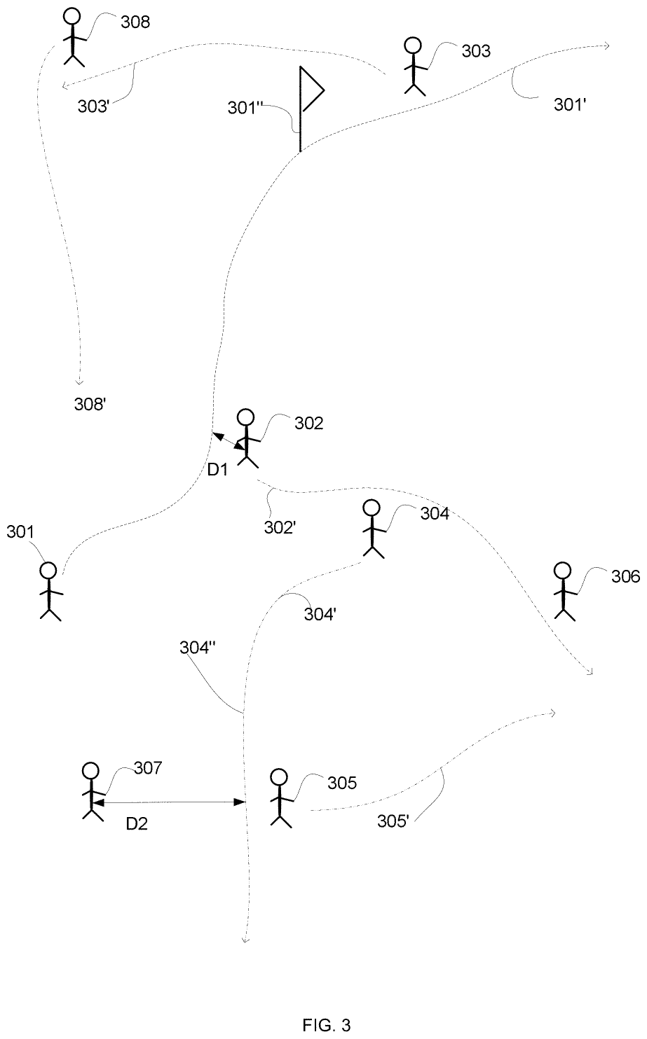

FIG. 3 illustrates a first suspected person 301, other suspected persons and a non-suspected person.

First till eighth persons 301-308 follow first till eighth paths—out of which first till fifth paths and eighth path 301′-305′ and 308′ are shown.

The first suspected person 301 was detected at the start of first path 301′ but was identified at point 301″ along a first path 301.

Second person 302 was close enough (distance D1) to first person 301 and can be regarded as a suspected direct infected person.

Third person 303 was close enough to first person 301 and can be regarded as a suspected direct infected person.

Fourth person 304 was close enough to second person 302 and can be regarded as a suspected indirect infected person.

Fifth person 305 was close enough to fourth person 304 and can be regarded as a suspected indirect infected person.

Sixth person 306 was close enough to second person 302 and can be regarded as a suspected indirect infected person.

Seventh person 307 was not close enough (distance D2 that well exceeds a minimal distance) to fourth person 304 (and was not close enough to any other person) and can be regarded as an un-suspected person.

Eighth person 308 was close enough to third person 303 and can be regarded as a suspected indirect infected person.

The eight persons may be represented in sensed information caught by one or more sensors. If there are multiple sensors then their fields of view may overlap each other, may partially overlap each other, or may have no overlaps.

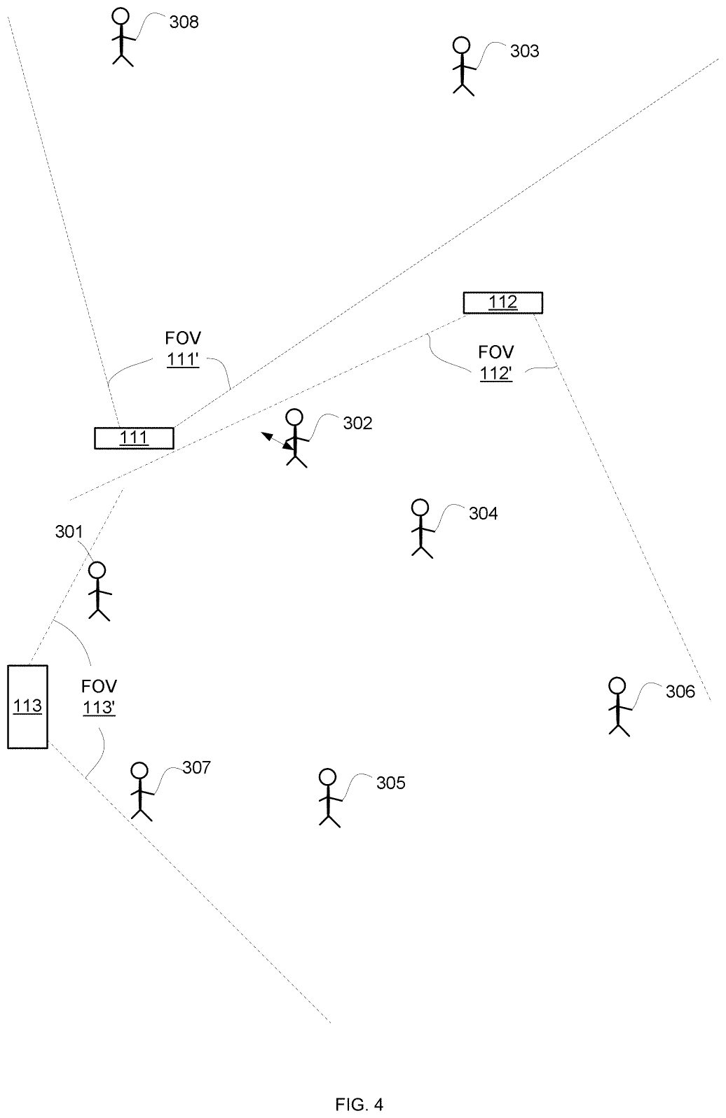

FIG. 4 illustrates that the first till eighth persons 301-308 are captured by three sensors.

Third and eighth persons (303 and 308) are within a first field of view 111′ of first sensor 111. At least second, fourth and sixth persons (302, 304 and 306) are within a second field of view 112′ of second sensor 112. At least first, fifth and seventh persons (301, 305 and 307) are within a third field of view 113′ of third sensor 113.

Any spatial and/or angular relationship may be provided between the fields of views of the different sensors.

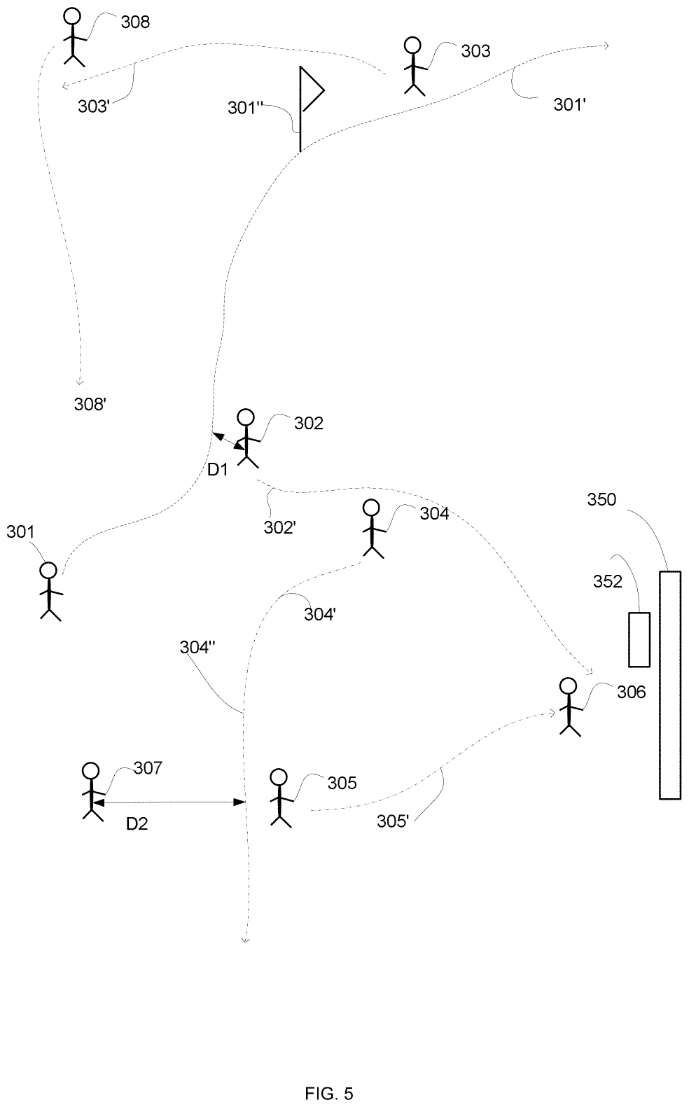

FIG. 5 illustrates a first suspected person 301, other suspected persons and a non-suspected person—that include first till eighth persons 301-308. In addition—there is an asset 350 and an access control entity 362 that may prevent, following an alert regarding a suspected person (for example sixth person 306)—to prevent the suspected person from entering asset 350.

The terms persons and people are used in an interchangeable manner.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Furthermore, the terms “assert” or “set” and “negate” (or “deassert” or “clear”) are used herein when referring to the rendering of a signal, status bit, or similar apparatus into its logically true or logically false state, respectively. If the logically true state is a logic level one, the logically false state is a logic level zero. And if the logically true state is a logic level zero, the logically false state is a logic level one.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It is appreciated that various features of the embodiments of the disclosure which are, for clarity, described in the contexts of separate embodiments may also be provided in combination in a single embodiment. Conversely, various features of the embodiments of the disclosure which are, for brevity, described in the context of a single embodiment may also be provided separately or in any suitable sub-combination.

It will be appreciated by persons skilled in the art that the embodiments of the disclosure are not limited by what has been particularly shown and described hereinabove. Rather the scope of the embodiments of the disclosure is defined by the appended claims and equivalents thereof.

What is claimed is:

1. A method for monitoring potential infection events, the method comprises:
obtaining sensed information gathered during a monitoring period;
identifying, within the sensed information, a first suspected person that is suspected to suffer from an infectious disease; wherein the identifying comprises applying a face recognition process on the sensed information;
detecting suspected persons that appear in the sensed information; wherein the detecting comprises finding suspected directly infected persons and finding suspected indirectly infected people; and
responding to the detecting of the suspected persons, wherein the responding comprises at least one out of generating an alert, transmitting an alert, storing an alert, and updating at least one data structure regarding at least one suspected person;
wherein each suspected directly infected person was involved in a suspected direct infection event related to the first suspected person; and wherein the finding of the suspected directly infected persons is based on distances between the first suspected person and other persons; and
wherein each indirectly infected people was involved in one or more other suspected infection events that are associated with the suspected direct infection events.

2. The method according to claim 1 wherein the wherein the finding of the suspected directly infected persons is based on distances between the first suspected person and other persons and on time periods in which the distances were maintained.

3. The method according to claim 1 wherein the searching for suspected directly infected persons comprises determining whether an identity of each suspected directly infected person is clear during an occurrence of a potential direct infection event; wherein when determining that the identity of a certain suspected directly infected person is not clear during an occurrence of a potential direct infection event than continuing to tracking after the certain suspected directly infected person until at least additional information for clarifying the identity is detected.

4. The method according to claim 1 wherein the searching for suspected directly infected persons comprises determining an occurrence of the one or more potential direct infection events related to the first suspected person based on contacts between the first suspected person and other persons.

5. The method according to claim 1 comprising obtaining the sensed information from multiple sensors.

6. The method according to claim 1 wherein the monitoring and the detecting is based on generating signatures of the sensed information.

7. The method according to claim 1 wherein the obtaining, identifying, detecting, and responding are executed in real time and wherein the responding comprises generating and transmitting, in real time, alerts to suspected persons.

8. The method according to claim 1 wherein the obtaining, identifying, detecting, and responding are executed in real time and wherein the responding comprises generating and transmitting, in real time, alerts that notify people located at a vicinity of the suspected persons, about the suspected persons.

9. The method according to claim 1 wherein the obtaining, identifying, detecting and responding are executed in real time and wherein the responding comprises generating and transmitting, in real time, alerts that notify at least one access control entity of at least one asset that is located at a vicinity of at least one suspected person, about the at least one suspected person.

10. The method according to claim 1 wherein the obtaining, identifying, detecting, and responding are executed in non-real time and wherein the responding comprises analyzing a distribution of a pandemic.

11. A non-transitory computer readable medium for configuring spanning elements of a signature generator, the non-transitory computer readable medium stores instructions for:
obtaining sensed information gathered during a monitoring period;
identifying, within the sensed information, a first suspected person that is suspected to suffer from an infectious disease; wherein the identifying comprises applying a face recognition process on the sensed information;
detecting suspected persons that appear in the sensed information, wherein the detecting of the suspected persons comprises:
finding suspected directly infected persons, each suspected directly infected person was involved in a suspected direct infection event related to the suspected person; wherein the finding of the suspected directly infected persons is based on distances between the first suspected person and other persons; and
finding suspected indirectly infected people that were involved in one or more other suspected infection events that are associated with the suspected direct infection events.

12. The non-transitory computer readable medium according to claim 11 wherein the wherein the finding of the suspected directly infected persons is based on distances between the first suspected person and other persons and on time periods in which the distances were maintained.

13. The non-transitory computer readable medium according to claim 11 wherein the searching for suspected directly infected persons comprises determining whether an identity of each suspected directly infected person is clear during an occurrence of a potential direct infection event; wherein when determining that the identity of a certain suspected directly infected person is not clear during an occurrence of a potential direct infection event than continuing to tracking after the certain suspected directly infected person until at least additional information for clarifying the identity is detected.

14. The non-transitory computer readable medium according to claim 11 wherein the searching for suspected directly infected persons comprises determining an occurrence of the one or more potential direct infection events related to the first suspected person based on contacts between the first suspected person and other persons.

15. The non-transitory computer readable medium according to claim 11 comprising obtaining the sensed information from multiple sensors.

16. The non-transitory computer readable medium according to claim 11 wherein the monitoring and the detecting is based on generating signatures of the sensed information.

17. The non-transitory computer readable medium according to claim 11 wherein the obtaining, identifying, detecting, and responding are executed in real time and wherein the responding comprises generating and transmitting, in real time, alerts to suspected persons.

18. The non-transitory computer readable medium according to claim 11 wherein the obtaining, identifying, detecting and responding are executed in real time and wherein the responding comprises generating and transmitting, in real time, alerts that notify at least one access control entity of at least one asset that is located at a vicinity of at least one suspected person, about the at least one suspected person.

19. The non-transitory computer readable medium according to claim 11 wherein the obtaining, identifying, detecting, and responding are executed in non-real time and wherein the responding comprises analyzing a distribution of a pandemic.

20. A method for monitoring potential infection events, the method comprises:
a sensed information gathering unit that is configured to gather sensed information during a monitoring period;
a processor that is configured to:
identify, within the sensed information, a first suspected person that is suspected to suffer from an infectious disease; wherein the identifying comprises applying a face recognition process on the sensed information;
detect suspected persons that appear in the sensed information; wherein the detecting comprises finding suspected directly infected persons and finding suspected indirectly infected people; and
a response unit that is configured to respond to the detecting of the suspected persons, wherein the responding comprises at least one out of generating an alert, transmitting an alert, storing an alert, and updating at least one data structure regarding at least one suspected person;
wherein each suspected directly infected person was involved in a suspected direct infection event related to the suspected person; and wherein the finding of the suspected directly infected persons is based on distances between the first suspected person and other persons; and
wherein each indirectly infected people was involved in one or more other suspected infection events that are associated with the suspected direct infection events.
